(19) United States
(12) Patent Application Publication
(10) Pub. No.: US 2012/0302915 A1
(43) Pub. Date: Nov. 29, 2012

(54) SPIRAL BIOPSY NEEDLE

(76) Inventor: Jonathon T. Lee, Flossmoor, IL (US)

(21) Appl. No.: 13/481,233

(22) Filed: May 25, 2012

Related U.S. Application Data
(60) Provisional application No. 61/490,847, filed on May 27, 2011.

(51) Int. Cl. A61B 10/02

(52) U.S. Cl. 600/567

(57) ABSTRACT

A spiral biopsy needle includes an elongated outer sleeve. A sampling tool having a penetrating tip, a grasping region and a helical thread collection region adjacent the tip is inserted into the outer sleeve. A spacer element is received on the tool between the grasping portion and the penetrating tip. When the sampling tool is inserted into the sleeve with the spacer element in place the penetrating tip extends beyond the outer sleeve end, but the collection region does not extend beyond the end. When the spacer element is removed and the sampling tool is further inserted into the sleeve, the sampling tool collection region extends beyond the outer sleeve free end for collection of a tissue sample. A method for obtaining a tissue sample is also disclosed.

### CROSS-REFERENCE TO RELATED APPLICATION DATA

This application claims the benefit of priority of Provisional U.S. Patent Application Ser. No. 61/490,847, filed May 27, 2011, the disclosure of which is incorporated herein by reference in its entirety.

### BACKGROUND

Two principal types of biopsy needles are known. Such needles are utilized for solid organ biopsies under computed tomography (CT) or ultrasound guidance. One type of needle is a core biopsy needle which removes chunks of tissue within a chamber grooved into the needle.

The second type of needle is an aspiration biopsy needle and is used mainly for cytologic samples. There are several variations of aspiration biopsy needle. Each has a different cutting edge at the tip of the needle and use syringe generated suction to obtain a sample. During a given procedure, anywhere from 3-12 or more aspiration needles are utilized to obtain adequate tissue samples.

There are, however, a number of drawbacks to the known biopsy needles. At times, the cells obtained within the aspiration biopsy needle are inadequate for an accurate diagnosis. As such, multiple needles are utilized for a single procedure. Given that the cells are inadequate, sometimes a core biopsy needle has to be utilized to obtain tissue samples. This may lead to a greater risk for complications. The core needle tends to be larger in size and has a spring forward mechanism which can cause damage to surrounding organs.

Moreover, the aspiration portion of the biopsy is performed with a back and forth motion of the needle within the organ. Under ultrasound guidance, the needle can be directly observed during the biopsy. However, under CT guidance, the needle is not observed and can result in damage to adjacent structures. Due to the uncertainty of the aspirating motion, adequate placement of the needle cannot be ensured during the biopsy itself. Therefore, lesions near critical organs may not be biopsied safely. Certain lesions can be very vascular. In these instances, the suction motion draws back mostly blood and can result in inadequate sampling of tumor cells.

Accordingly, there is a need for an improved biopsy needle. Such a needle permits accurately establishing an insertion depth at which samples are drawn from tissue. Desirably, such a needle system can be adjusted to ensure that new samples can be taken from slightly different portions of a lesion. More desirably still, the needle system can be moved together to other areas of the target lesion prior to sampling, which is seen to be particularly beneficial in examination of larger lesions.

### BRIEF SUMMARY

A spiral biopsy needle has an elongated outer sleeve, a sampling tool having a penetrating tip, a grasping region opposite the tip, and a collection region between the penetrating tip and the grasping region. The sampling tool is insertable into the outer sleeve and a spacer element is positioned on the sampling tool between the penetrating tip and the grasping region.

The sleeve has a free end and an insertion end and defines a length. Preferably, the sleeve includes a collar thereon, at the insertion end.

The sampling tool has an elongated body for insertion into the insertion end of the outer sleeve. The grasping portion has a stop surface. Preferably, the collection region is defined by a helical spiral thread formed on the tool adjacent the penetrating tip.

The spacer element is received on the tool body between the grasping portion and the penetrating tip. The spacer element, when in place, engages the collar and the grasping region and locks the sampling tool from further penetration.

When the sampling tool is inserted into the outer sleeve with the spacer element in place, the penetrating tip extends beyond the outer sleeve free end. When the spacer element is removed and the sampling tool is further inserted into the outer sleeve, the sampling tool collection region extends beyond the outer sleeve free end.

Preferably, when the sampling tool is inserted into the outer sleeve with the spacer element in place on the sampling tool and between the grasping portion and the insertion end, the collection region resides fully within the outer sleeve.

The grasping region can be configured as a thumb wing having a flat surface on a bottom thereof that abuts the spacer element when the spacer element is in place.

The penetrating tip can have a diameter less than or about equal to a major diameter of the thread. The thread can be symmetrical or asymmetrical. The pitch of the thread can be substantially constant along a threaded length of the collection region or it can vary.

The free end of the outer sleeve can taper toward a longitudinal axis of the outer sleeve.

The spacer element is a member having a predetermined length and can include a slotted opening along its length for positioning the spacer element on the sampling tool body and removing the spacer element from the sampling tool body when the sampling tool is inserted in the outer sleeve.

A method for obtaining a sample from tissue in a body includes the steps of positioning a spiral biopsy needle in the body adjacent the tissue to be sampled, the spiral biopsy needle having an outer sleeve having a free end and an insertion end, the spiral biopsy needle further including a sampling tool positioned in the outer sleeve, the sampling tool having a penetrating tip at a free end thereof, a grasping portion at an end opposite the free end and having a collection region adjacent the penetrating tip, the spiral biopsy needle further including a spacer element configured for receipt on the sampling tool body between the grasping portion and the penetrating tip. The method further includes removing the spacer element from the sampling tool body, urging the sampling tool forward into the body while maintaining the outer sleeve in place by rotating the sampling tool into the tissue to collect tissue in the collection region and withdrawing the sampling tool from the outer sleeve.

The method can include the step of reinserting the sampling tool in the outer sleeve and obtaining a subsequent sample of tissue. This can include the step of repositioning the spiral biopsy needle in the body prior to obtaining in the subsequent sample of tissue. The method can further include the step of inserting a different sampling tool in the outer sleeve and obtaining a subsequent sample of tissue.

Other objects, features, and advantages of the disclosure will be apparent from the following description, taken in conjunction with the accompanying sheets of drawings, wherein like numerals refer to like parts, elements, components, steps, and processes.

### BRIEF DESCRIPTION OF THE DRAWINGS

Details of the present disclosure, including non-limiting benefits and advantages, will become more readily apparent to those of ordinary skill in the relevant art after reviewing the following detailed description and accompanying drawings, wherein:

FIG. 1 is a perspective view of an embodiment of a spiral biopsy needle;

FIGS. 2A-2C are perspective, side-sectional and top views of the outer sleeve and collar;

FIG. 3 is a sectional view of the outer sleeve of FIG. 2;

FIG. 4 is a sectional view of and alternate outer sleeve;

FIGS. 5A-5C are perspective, side and top views of an embodiment of a sampling tool;

FIGS. 6A-6D are various views of the tool shaft of FIG. 5, shown in a side view (6A), partial perspective view (6B), and side-sectional detail views (6C and 6D);

FIGS. 7A-7D are various views of an alternate embodiment of the tool shaft, shown in a side view (7A), partial perspective view (7B), and side-sectional detail views (7C and 7D);

FIG. 8A-8C are various views of still another alternate embodiment of the tool, shown in a side view (8A), partial perspective view (8B), and detail view (SC);

FIG. 9 is a side view of an solid shaft sampling tool;

FIGS. 10A-10C are perspective, side and top views, respectively, of the shaft spacer element;

FIGS. 11A-11F are various thread configurations on the tool shaft; and

FIGS. 12A and 12B illustrate the spiral biopsy needle in the insert and sampling configurations.

### DETAILED DESCRIPTION

While the present disclosure is susceptible of embodiment in various forms, there is shown in the drawings and will hereinafter be described an embodiment with the understanding that the present disclosure is to be considered an example and is not intended to limit the disclosure to the specific embodiment illustrated.

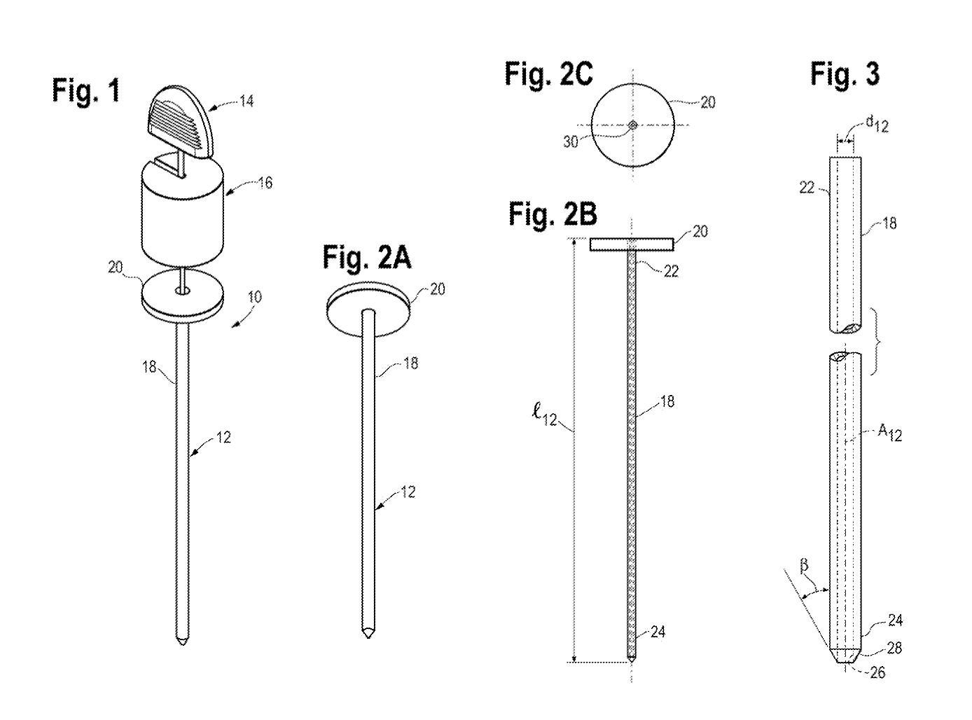

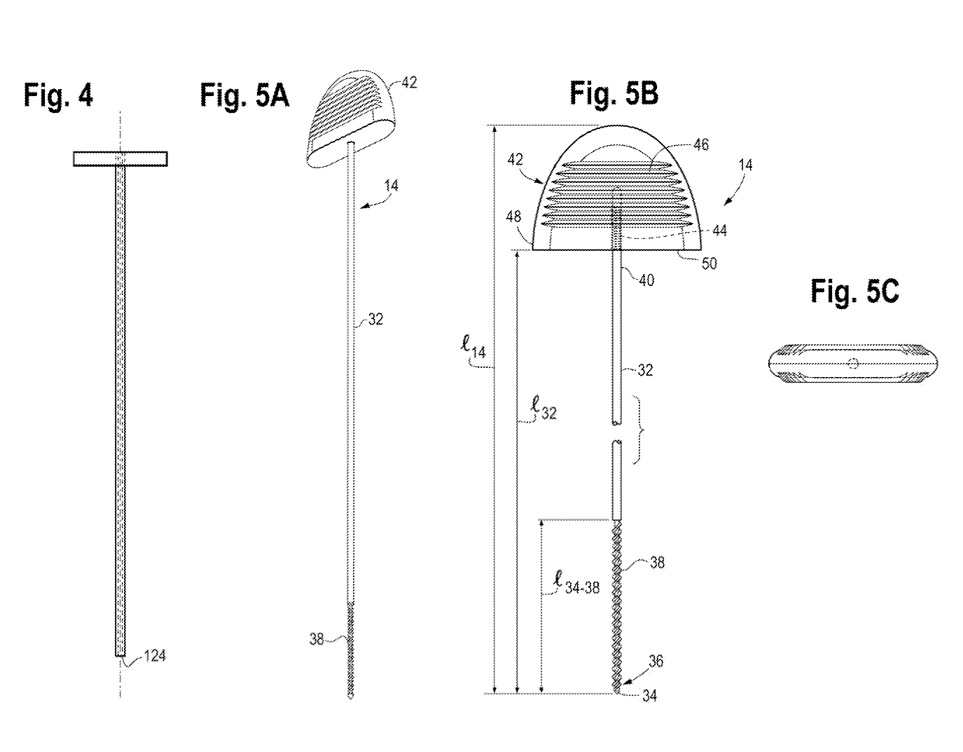

Referring now to the figures and in particular to FIG. 1, there is shown a spiral biopsy needle 10. The needle 10 includes, generally, an outer sleeve assembly 12, a sampling tool 14 and a spacer element 16. As seen in FIGS. 2A-2C and 3, the outer sleeve assembly 12 includes an outer sleeve tube 18 and a sleeve collar 20. The outer sleeve tube 18 is an elongated hollow member having a first end 22 at the collar 20 and a free end 24. The hollow member defines a bore 26 having a predetermined diameter d₂₆. In one embodiment, the free end 24 has a taper (as indicated at 28) toward the free end 24 of the tube 18. The taper 28 can be formed at an angle β of about 30 degrees to the axis A₁₂. Alternately, as seen in FIG. 4, the free end 124 can be squared relative to a longitudinal axis A₁₂ of the sleeve 12.

The collar 20 is a circumferentially enlarged flange that is affixed to the collar end 22 of the tube 18. The collar 20 has a bore 30 into which the tube 18 fits such that the opening 26 in the tube 18 at the collar end 22 is accessed through the collar opening 30. Alternately, the collar can be fitted to the tube such that the collar opening and the tube bore are contiguous with one another.

FIGS. 5A-5C illustrate one exemplary sampling tool 14. The tool 14 includes an elongated shaft 32 having a penetrating tip 34 on a free end 36 thereof and a helical thread formation 38 adjacent or formed as part of the penetrating tip 34. An opposing end 40 of the shaft 32 has a handle 42, such as the illustrated thumb wing, mounted thereto. The thumb wing 42 can be mounted to the shaft 32 by, for example, machined threads 44 on the shaft that cooperate with an opening in the thumb wing 42. Other manners in which the thumb wing 42 and shaft 32 can be joined to one another, such as by adhesive or even integral formation (e.g., a one-piece shaft and thumb wing) will be appreciated by those skilled in the art. As illustrated the thumb wing 42 can include a portion 46 thereon that permits securely grasping and manipulating the sampling tool 14. In a preferred embodiment, the base or bottom 48 of the thumb wing 42, that is, that portion that is proximal the shaft 32, has a flat surface as indicated at 50.

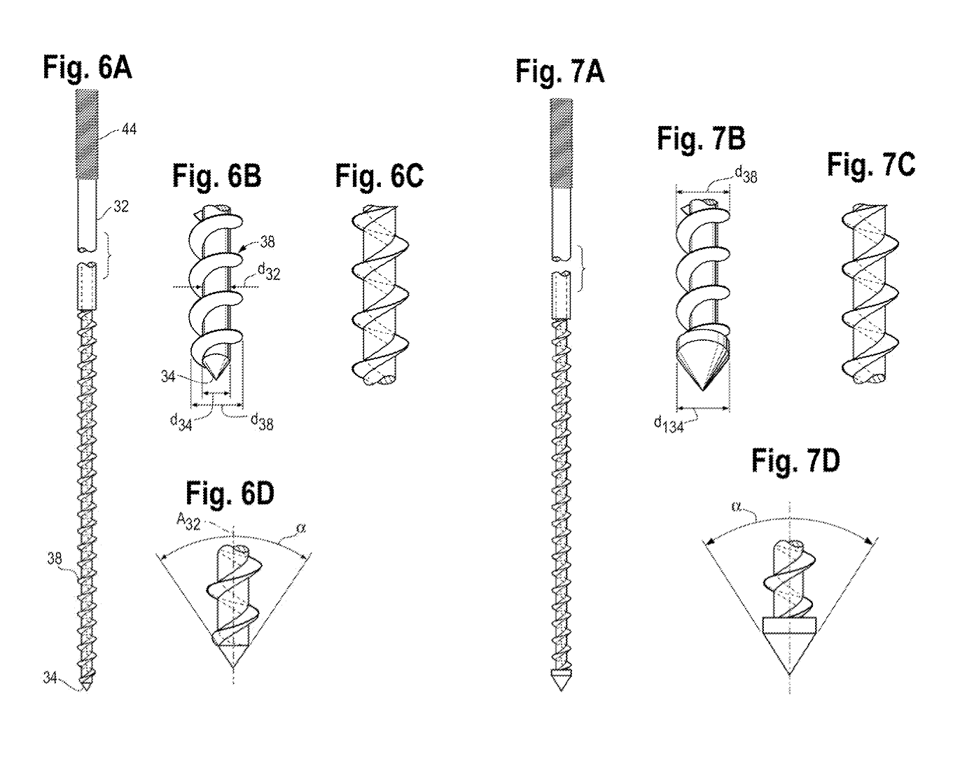

The penetrating tip 34 can be formed as a pointed conical member sufficiently sharp to penetrate the skin and any tissue from which a sample will be taken. The tip 34 can have a maximum diameter d₃₄ that is smaller than the major diameter d₃₈ of the adjacent thread 38 (see, for example, FIGS. 6A and 6B), or it can have a maximum diameter d₁₃₄ that is about the same size as the major diameter d₃₈ of the adjacent thread 38 (see, for example, FIGS. 7A and 7B).

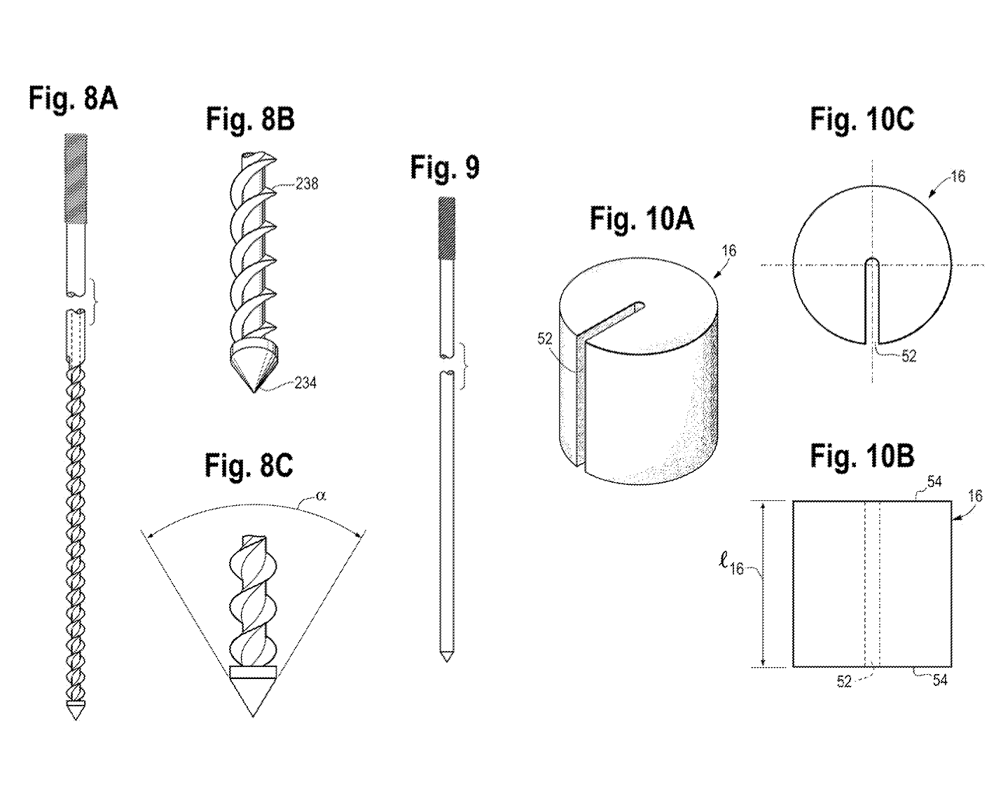

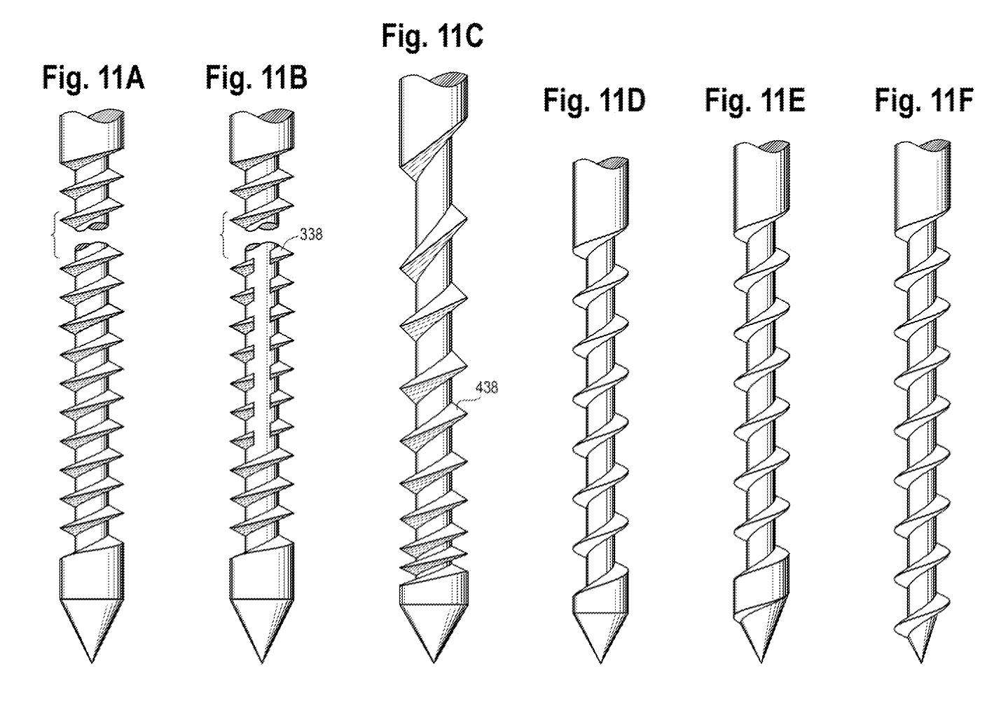

The thread 38 can also have various formations and configurations. The thread can be a symmetrical thread (e.g., mirror image thread flanks as seen in FIGS. 6 and 7), or asymmetrical, with one flank being concave or cupped 238 (as seen in FIGS. 8A-8C). Other thread 38 formations can include a discontinuous thread 338 (as seen in FIG. 11B), or a thread 438 having a pitch that changes along the length of the thread (as seen in FIG. 11C). Both coarse and fine thread pitches can also be used. Small grooves or divots can be placed in the thread or in the shaft, between the threads, to capture more tissue. In all, as discussed below, the function of the threads (38, collectively) is to capture tissue sample within the thread 38.

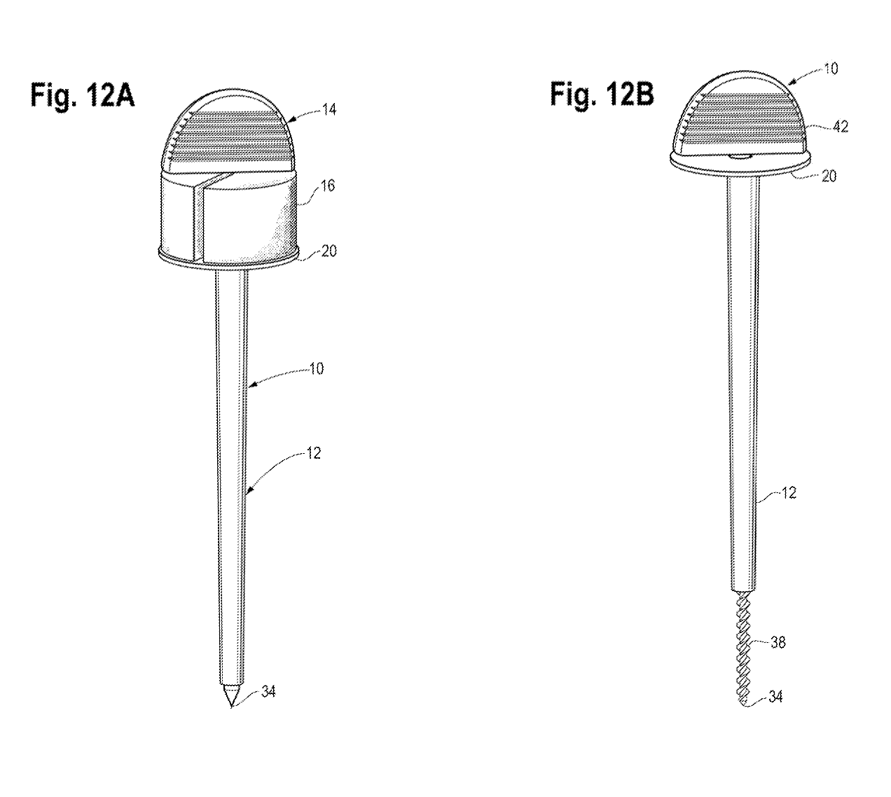

The needle 10 further includes a spacer element 16. The spacer element 16, when used, is positioned between the collar 20 and the thumb wing 42. The spacer element 16 has a slotted opening 52 or cut-out formed therein that permits the spacer element 16 to be slipped onto the shaft 32. In a present embodiment, the spacer element 16 has flat opposing surfaces 54 that, when the needle 10 is assembled, as see in FIG. 12A, abut the collar 20 on one end and the thumb wing base 50 on the other end. A present spacer element 16 has a cylindrical shape that complements the shape and size of the collar 20.

As will be discussed below, the spacer element 16 has a length l₁₆ that, when in place between the thumb wing 42 and collar 20, permits the penetrating tip 34 to extend slightly beyond the free end 24 of the sleeve 12. The spacer element 16 serves as a lock to retain the tool 14 in place in the sleeve 12.

The relative diameters of the tool shaft (the major diameter d₃₈ of the thread 38) and the inside diameter d₁₂ of the sleeve 12 (i.e., the diameter of the bore 26) are such that the tool 14 can be readily inserted into and withdrawn from the sleeve 12 without a great deal of force, but with only minimal clearance between the thread 38 and the inside wall of the sleeve 12. In one tool 14, the thread 38 has a major diameter d₃₈ of about 1.0 mm and a minor diameter d₃₂ (the shaft 32 diameter) of about 0.5 mm and the sleeve 12 has an inside diameter d₁₂ of about 1.0 mm. The angle α formed by the penetrating tip 34 is about 30 degrees relative to a longitudinal axis A₃₂ of the shaft 32. The shaft 32 has an overall length l₃₂ of about 125 mm with the penetrating tip 34 and thread 38 having a length l₃₄₋₃₅ of about 20-21 mm and the sampling tool 14, including the thumb wing 42 has an overall length l₁₄ of about 140 mm. The sleeve 12 has a length l₁₂ of about 104 mm including the collar 20. The spacer element 16 has a length l₁₆ of about 20 mm.

In one contemplated use, the needle 10 is in an assembled state with the sampling tool 14 inserted into the sleeve 12 and the spacer element 16 positioned on the shaft 32 between the thumb wing 42 and the collar 20. This is the insertion configuration as seen in FIG. 12A. The penetrating tip 34 will extend slightly from the sleeve free end 24. It is anticipated that in this configuration, the thread 38 is not exposed from the sleeve 12.

The needle 10 is introduced (inserted) into the body and positioned at a target lesion. The spacer element 16 remains in place until the needle 10 is located at the periphery of the lesion. When the penetrating tip 34 reaches the target lesion, the spacer element 16 is removed and the sampling tool 14 is screwed (threaded) into the target to the exact location needed.

After confirming the exact position of the tip 34, the tool 14 is retracted through the sleeve 12. The sleeve 12 can be left in place at the periphery of the lesion for a second sampling to be performed if desired. If a subsequent sample is to be taken, the tool 14 can be reinserted, if, for example, it is rinsed in saline, and the subsequent sample can be obtained. Alternately a second tool 14 can be inserted into the sleeve 12 to obtain the subsequent sample, if, for example, the first tool is rinsed in formalin.

Once adequate samples are obtained, the tool 14 can be reintroduced into the sleeve 12 and locked in place (using the spacer element 16) and the coaxial needle system 10 can be removed safely from the body.

Those skilled in the art will recognize that the present spiral biopsy needle system 10 provides significant advantages over known biopsy needles or systems. First, the cells trapped within the spiral thread 38 can be given to a pathologist for a touch prep as well as rinsed within saline or formalin for formation of a cell block. Second, the insertion depth of the needle system 10 can be adjusted to ensure that new samples are from a slightly different portion of the lesion. Moreover, the coaxial needle system 10 can also be moved together to another area of the target lesion prior to sampling. This will be particularly beneficial in larger lesions.

In the present disclosure, the words “a” or “an” are to be taken to include both the singular and the plural. Conversely, any reference to plural items shall, where appropriate, include the singular. All patents referred to herein, are hereby incorporated herein by reference, whether or not specifically done so within the text of this disclosure.

Numerous modifications to the present disclosure will be apparent to those skilled in the art in view of the foregoing description. Accordingly, this description is to be construed as illustrative only and is presented for the purpose of enabling those skilled in the art to make and use the invention and to teach the best mode of carrying out same. The exclusive rights to all modifications which come within the scope of the appended claims are reserved.

1. A spiral biopsy needle, comprising:
an elongated outer sleeve defining a bore longitudinally therein, the outer sleeve having a free end and an insertion end defining a length;
a sampling tool, the sampling tool having an elongated body configured for insertion into the insertion end of the outer sleeve, the sampling tool having a penetrating tip at a free end thereof and having a grasping portion at an opposite end thereof, the grasping portion having a stop surface, the elongated body defining a length longer than the length of the outer sleeve, the sampling tool having a collection region adjacent the penetrating tip;
a spacer element configured for receipt on the tool body between the grasping portion and the penetrating tip, the spacer element having a length,
wherein when the sampling tool is inserted into the outer sleeve with the spacer element in place on the sampling tool and between the grasping portion and the insertion end, the penetrating tip extends beyond the outer sleeve free end, and when the spacer element is removed and the sampling tool further inserted into the outer sleeve, the sampling tool collection region extends beyond the outer sleeve free end.

2. The spiral biopsy needle of claim 1 wherein when the sampling tool is inserted into the outer sleeve with the spacer element in place on the sampling tool and between the grasping portion and the insertion end, the collection region resides fully within the outer sleeve.

3. The spiral biopsy needle of claim 1 wherein the collection region is defined by a helical thread formation on the elongated body.

4. The spiral biopsy needle of claim 1 including a collar on the outer sleeve at the insertion region and wherein the spacer element, when in place, engages the collar and the grasping region.

5. The spiral biopsy needle of claim 1 wherein the grasping region is a thumb wing.

6. The spiral biopsy needle of claim 5 wherein the thumb wing has a flat surface on a bottom thereof, the flat surface configured to abut the spacer element when the spacer element is in place.

7. The spiral biopsy needle of claim 3 wherein the penetrating tip has a diameter about equal to a major diameter of the thread.

8. The spiral biopsy needle of claim 3 wherein the penetrating tip has a diameter less than a major diameter of the thread.

9. The spiral biopsy needle of claim 3 wherein the thread is symmetrical.

10. The spiral biopsy needle of claim 3 wherein a pitch of the thread is substantially constant along a threaded length of the collection region.

11. The spiral biopsy needle of claim 1 wherein the free end of the outer sleeve is tapered toward a longitudinal axis of the outer sleeve.

12. The spiral biopsy needle of claim 1 wherein the spacer element is a member having a length and defining a slotted opening along a length thereof for positioning the spacer element on the sampling tool elongated body and removing the spacer element from the sampling tool elongated body when the sampling tool is inserted in the outer sleeve.

13. A method for obtaining a sample from tissue in a body comprising the steps of:
positioning a spiral biopsy needle in the body adjacent the tissue to be sampled, the spiral biopsy needle having an outer sleeve having a free end and an insertion end, the spiral biopsy needle further including a sampling tool positioned in the outer sleeve, the sampling tool having a penetrating tip at a free end thereof, a grasping portion at an end opposite the free end and having a collection region adjacent the penetrating tip, the spiral biopsy needle further including a spacer element configured for receipt on the sampling tool body between the grasping portion and the penetrating tip;
removing the spacer element from the sampling tool body;
urging the sampling tool forward into the body while maintaining the outer sleeve in place by rotating the sampling tool into the tissue to collect tissue in the collection region; and
withdrawing the sampling tool from the outer sleeve.

14. The method of claim 13 including the step of reinserting the sampling tool in the outer sleeve and obtaining a subsequent sample of tissue.

15. The method of claim 14 including the step of repositioning the spiral biopsy needle in the body prior to obtaining in the subsequent sample of tissue.

16. The method of claim 13 including the including the step of inserting a different sampling tool in the outer sleeve and obtaining a subsequent sample of tissue.

17. The method of claim 16 including the step of positioning the spiral biopsy needle in the body prior to obtaining the subsequent sample of tissue.
